(19) United States
(12) Patent Application Publication
(10) Pub. No.: US 2008/0077247 A1
(43) Pub. Date: Mar. 27, 2008

(54) Spinal spacer

(75) Inventors: Fred Murillo, San Diego, CA (US); Mark Melton, Carlsbad, CA (US)

Correspondence Address: MINTZ LEVIN COHN FERRIS GLOVSKY & POPEO, 666 THIRD AVENUE, NEW YORK, NY, 10017, US

(73) Assignee: ALPHATEC SPINE, INC., Carlsbad, CA (US)

(21) Appl. No.: 11/903,895

(22) Filed: Sep. 24, 2007

Related U.S. Application Data
(60) Provisional application No. 60/846,568, filed on Sep. 22, 2006.

(51) Int. Cl. A61F 2/44

(52) U.S. Cl. 623/17.16

(57) ABSTRACT

A spinal spacer assembly is provided. The spinal spacer assembly includes a spinal spacer having a wall configured to enclose a hollow interior, wherein the wall is further configured to have a top portion and a bottom portion. The top and bottom portions are configured to include a plurality of protrusions configured to protrude away from the top and bottom portions. The wall is further configured to have a variable degree curvature and include a plurality of grooves and an opening.

### CROSS-REFERENCE TO RELATED APPLICATIONS

The present application claims priority to U.S. Provisional Patent Application No. 60/846,568 to Murillo et al., filed Sep. 22, 2006, and entitled “Spinal Spacer”, the disclosure of which is incorporated herein by reference in its entirety.

### BACKGROUND OF THE INVENTION

1. Field of the Invention

The present invention is directed to systems, methods, and devices applicable to spinal surgery. More specifically, the present invention is directed to a spinal spacer for use by medical personnel (i.e., doctor) in spinal and other surgical procedures. In some embodiments of the present invention relates to a spinal spacer for insertion into a disk space defined between two adjacent vertebrae, in order to restore an appropriate height between the vertebrae and to allow bone fusion to take place between said adjacent vertebrae.

2. Background of the Invention

Vertebrae are the individual irregular bones that make up the spinal column (aka ischis)—a flexuous and flexible column. There are normally thirty-three vertebrae in humans, including the five that are fused to form the sacrum (the others are separated by intervertebral discs) and the four coccygeal bones which form the tailbone. The upper three regions comprise the remaining 24, and are grouped under the names cervical (7 vertebrae), thoracic (12 vertebrae) and lumbar (5 vertebrae), according to the regions they occupy. This number is sometimes increased by an additional vertebra in one region, or it may be diminished in one region, the deficiency often being supplied by an additional vertebra in another. The number of cervical vertebrae is, however, very rarely increased or diminished.

A typical vertebra consists of two essential parts: an anterior (front) segment, which is the vertebral body; and a posterior part—the vertebral (neural) arch—which encloses the vertebral foramen. The vertebral arch is formed by a pair of pedicles and a pair of laminae, and supports seven processes, four articular, two transverse, and one spinous, the latter also being known as the neural spine.

When the vertebrae are articulated with each other, the bodies form a strong pillar for the support of the head and trunk, and the vertebral foramina constitute a canal for the protection of the medulla spinalis (spinal cord), while between every pair of vertebrae are two apertures, the intervertebral foramina, one on either side, for the transmission of the spinal nerves and vessels.

Conventional spinal spacer assemblies are use in spinal fusion procedures to repair damaged or incorrectly articulating vertebrae. Spinal fusion employs the use of spacer assemblies having a hollow mesh spacer tube and end caps that space apart and fuse together adjacent vertebrae. These mesh spacer tubes are often formed of titanium and are available in varying shapes and sizes. In addition, they can be trimmed on site by the surgeon to provide a better individual fit for each patient. Conventional spinal spacer assemblies come in different cross sections. These spacer assemblies are generally hollow and include openings in the side thereof to provide access for bone to grow and fuse within the mesh tube.

There exists a need for further improvements in the field of spinal spacer assemblies of the present type.

### SUMMARY OF THE INVENTION

In some embodiments, the present invention relates to a spinal spacer. The spinal spacer having a wall configured to enclose a hollow interior, wherein the wall is further configured to have a top portion and a bottom portion. The top and bottom portions are configured to include a plurality of protrusions or teeth configured to protrude away from the top and bottom portions. The wall is further configured to have at least one curved portion (or curvature) and include a plurality of grooves and an opening.

In some embodiments, the present invention relates to a spinal spacer assembly. The assembly includes a spinal spacer. The spinal spacer has a wall having two sides, a front portion, and a back portion, wherein the front and back portions are configured to be disposed between the two sides. The front and back portions are configured to include at least one curved portion. At least one side of the two sides includes at least one groove and at least one opening. The two sides and the front and back portions are configured to enclose a hollow interior. The wall is further configured to have a top surface and a bottom surface. The top and bottom surfaces are configured to include a plurality of protrusions configured to protrude away from the top and bottom portions. In some embodiments, the wall may have a variable thickness.

Further features and advantages of the invention, as well as structure and operation of various embodiments of the invention, are disclosed in detail below with references to the accompanying drawings.

### BRIEF DESCRIPTION OF THE DRAWINGS

The present invention is described with reference to the accompanying drawings. In the drawings, like reference numbers indicate identical or functionally similar elements.

FIG. 1A is a perspective view of an exemplary spinal spacer, according to some embodiments of the present invention.

FIG. 1B is a bottom view of the exemplary spinal spacer shown in FIG. 1A.

FIG. 1C is a top view of the exemplary spinal spacer shown in FIG. 1A.

FIG. 1D is a side view of the exemplary spinal spacer shown in FIG. 1A.

FIG. 1E is a cross-sectional view of the exemplary spinal spacer shown in FIG. 1A.

FIG. 1F is another side view of the exemplary spinal spacer shown in FIG. 1A.

FIG. 1G is another top view of the exemplary spinal spacer shown in FIG. 1A.

FIG. 1H is yet another side view along with a cross-section view of the exemplary spinal spacer shown in FIG. 1A.

FIG. 2A is a perspective view of another exemplary spinal spacer, according to some embodiments of the present invention.

FIG. 2B is a bottom view of the exemplary spinal spacer shown in FIG. 2A.

FIG. 2C is a top view of the exemplary spinal spacer shown in FIG. 2A.

FIG. 2D is a side view of the exemplary spinal spacer shown in FIG. 2A.

FIG. 2E is a cross-sectional view of the exemplary spinal spacer shown in FIG. 2A.

FIG. 2F is another side view of the exemplary spinal spacer shown in FIG. 2A.

FIG. 2G is another top view of the exemplary spinal spacer shown in FIG. 2A.

FIG. 2H is yet another side view along with a cross-section view of the exemplary spinal spacer shown in FIG. 2A.

FIG. 3A is a perspective view of another exemplary spinal spacer, according to some embodiments of the present invention.

FIG. 3B is a side view of the exemplary spinal spacer shown in FIG. 3A.

FIG. 3C is a top view and a cross-sectional view of the exemplary spinal spacer shown in FIG. 3A.

FIG. 3D is a side view and a cross-sectional view of the exemplary spinal spacer shown in FIG. 3A.

FIG. 3E is another side view of the exemplary spinal spacer shown in FIG. 3A.

FIG. 3F is another top view of the exemplary spinal spacer shown in FIG. 3A.

FIG. 3G is another top view of the exemplary spinal spacer shown in FIG. 3A.

FIG. 4A is a perspective view of another exemplary spinal spacer, according to some embodiments of the present invention.

FIG. 4B is a side view of the exemplary spinal spacer shown in FIG. 4A.

FIG. 4C is a top view and a cross-sectional view of the exemplary spinal spacer shown in FIG. 4A.

### DETAILED DESCRIPTION OF THE INVENTION

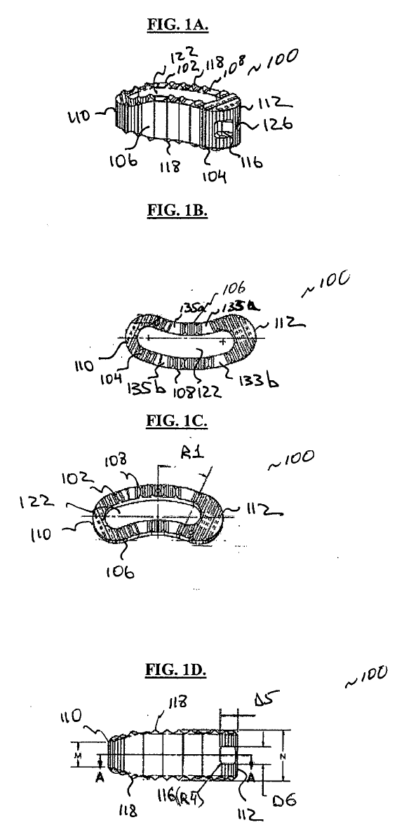

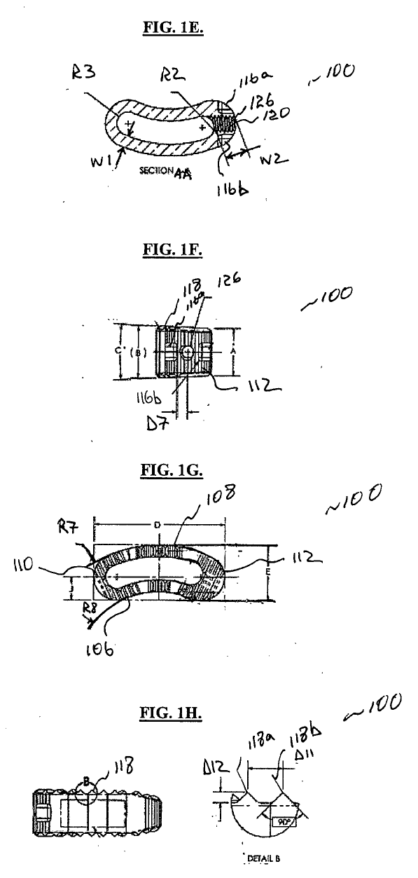

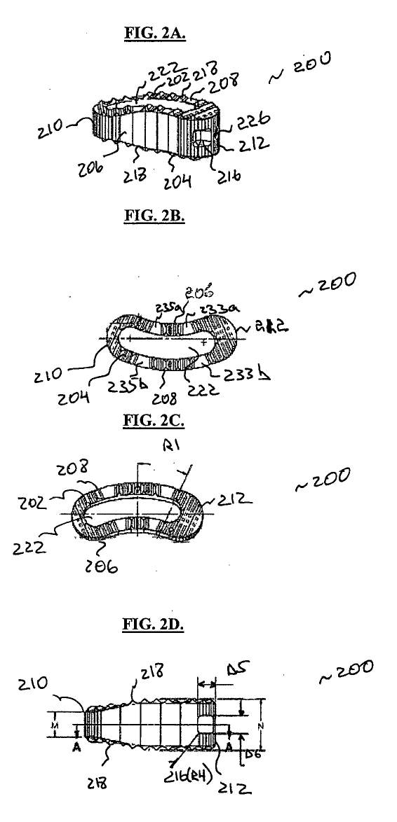

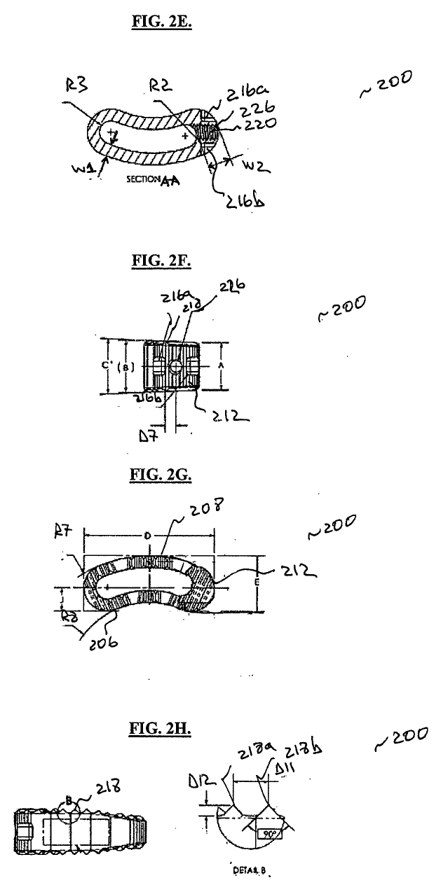

An exemplary embodiment of the spinal spacer, according to the present invention is illustrated in FIGS. 1A-1H. Another exemplary embodiment of the spinal spacer is illustrated in FIGS. 2A-2H. As can be understood by one skilled in the art, these embodiments are shown for illustrative purposes and are not intended to limit the scope of the invention.

FIGS. 1A-1H illustrate a spinal spacer 100 that includes a top portion 102, a bottom portion 104, a front side 106, a back side 108, a left side 110, and a right side 112. The front side 106, the back side 108, the left side 110 and the right side 112 may have a varying height, length, thickness, and/or curvature radius, as illustrated in FIGS. 1A-1H. As further illustrated in FIGS. 1A-1H, the sides 106, 108, 110, and 112 are configured to include at least one curved portion that can be configured to have a variable degree curvature radius.

As shown in FIG. 1A, a combination of the sides 106, 108, 110, and 112 forms a wall that encloses a hollow interior 122. The top portion 102 and the bottom portion 104 include a plurality of protrusions or teeth 118 (hereinafter, referred to as “teeth”). Teeth 118 can be configured to be spaced throughout the top portion 102 and the bottom portion 104. As can be understood by one skilled, the teeth 118 can be configured to have variable thickness, height, and width as well as angles of orientation with respect to surfaces of portions 102 and 104. The teeth 118 can be further configured to provide additional support after the spinal spacer 100 is implanted in the vertebrae of the patient. The teeth 118 can reduce movement of the spinal spacer 100 in the vertebrae and create additional friction between the vertebrae and the spacer 100. If more than one spinal spacer 100 is implanted in the vertebrae of the patient, the teeth 118 of one spinal spacer 100 can be configured to interact and/or mate with teeth of another spinal spacer, thereby creating stacked spacers. Such interaction can be useful, when multiple spinal spacers are needed to be implanted into the vertebrae. As shown in FIG. 1H (detail B of the side view of the spinal spacer 100), the teeth 118 can be configured to have a shape of triangular protrusions extending away from the surfaces of the top and bottom portions of the spinal spacer 100. The triangular protrusions can be configured to be right-angled isosceles triangles, as illustrated in detail B of FIG. 1H. As can be understood by one skilled in the art, the triangular protrusions can be any size and shape triangles are not necessarily limited to the right-angled isosceles triangles. Further, the triangular protrusions can be configured to protrude a distance D12 away from the surface (whether top or bottom surfaces) of the spinal spacer 100. The triangular protrusions can also be spaced apart a distance D11, as illustrated in detail B of FIG. 1H. In some embodiments, D12=0.03 millimeters (“mm”) and D11=0.1 mm. As can be understood by one skilled in the art, the teeth 118 can be configured to have any shape, size, or orientation as well as can protrude any distance away from the surfaces of the spinal spacer and can have any distance between them.

Referring back to FIGS. 1A-C, in some embodiments, the teeth 118 can be configured to be evenly spaced on the top portion 102 and the bottom portion 104. In other embodiments, the teeth 118 can be configured to be spaced in a predetermined order, such as the one shown in FIGS. 1A-C.

FIG. 1B is a bottom view of the spinal spacer 100 shown in FIG. 1A. The teeth 118 are configured to be disposed on the bottom portion 104 in a predetermined order. Specifically, the teeth 118 include a plurality of spacings 135(a, b) and 133(a, b). As illustrated in FIG. 1B, spacings 133a and 135a are configured to be disposed between teeth 118 on the bottom face 104 adjacent the front portion 106 of the spinal spacer 100. The spacings 133b and 135b are configured to be disposed between teeth 118 on the bottom face 104 adjacent the back portion 108 of the spinal spacer 100. Each spacing 133 and 135 is configured to be disposed at predetermined angles with regard to each other. In particular, spacings 133a and 133b are configured to be disposed at angles substantially matching a curvature of the front and back portions 106 and 108 of the spinal spacer 100. Similarly, spacings 135a and 135b are also configured to be disposed at angles substantially matching a curvature of the portions 106, 108. Further, the angular directions of spacings 133(a, b) and spacings 135(a, b) are configured to point away from each other, as illustrated in FIG. 1B. Such disposition allows the spinal spacer to more closely match the shapes and sizes of the vertebrae and accommodate other spinal spacers 100 in the event that spinal spacers are stacked together.

As further illustrated in FIG. 1B, some teeth 118 can have a different length than the other teeth 118. For example, teeth located on the bottom surface 104 adjacent the right side 112 and the left side 110 can be configured to be longer than the teeth located on the bottom surface 104 adjacent the front and back portions 106, 108.

FIG. 1C is a top view of the exemplary spinal spacer 100 shown in FIG. 1A. The top surface 102 also includes a plurality of teeth 118 that can be configured to have a similar structure as shown in FIG. 1H. The teeth 118 can be disposed through the top surface 102 in a similar fashion as their counterparts in the bottom surface 104. The teeth disposition can be substantially symmetrical about a center axis of the spacer 100. As can be understood by one skilled in the art, such symmetrical disposition can be in the top surface 102 as well as in the bottom surface 104 of the spinal spacer 100. Further, as shown in FIGS. 1B and 1C, the wall formed adjacent to the right side 112 of the spinal spacer 100 can be configured to have a greater thickness than the walls formed adjacent to the front portion, back portion, and left side of the spacer 100. In some embodiments, the thicknesses of the front portion, back portion, and left side can be configured to be substantially the same. As illustrated in FIG. 1E, such thicknesses W1 can be on the order of 0.1 mm, whereas thickness W2 of the wall adjacent to the right side 112 can be on order of 0.19 mm. As can be understood by one skilled in the art, these numerical values are provided here for exemplary purposes only and are not intended to limit the present invention in any way. The front portion 106 and the back portion 108 can be further configured to have a convex/concave shape. The convexity/concavity of these portions can be further defined by a radius R1. In exemplary embodiments, R1=25°. The spacings 133 and 135 can be disposed between teeth 118 to substantially match such angular disposition. At least a portion of the teeth 118 can also be disposed along top and bottom surfaces in an angular direction that substantially matches radius R1. As can be understood by one skilled in the art, other radii can be used to define curvatures of the spinal spacer 100.

FIG. 1D is a side view of the exemplary spinal spacer 100 illustrated in FIG. 1A. As illustrated in FIG. 1D, the left side 110 of the spinal spacer 100 can be configured to have a lesser thickness M than the thickness N of the right side 112 of the spinal spacer 100. The right side 112 can be further configured to accommodate an opening 116. In some embodiments, the spacer 100 can be configured to include more than one opening 116. As illustrated in FIG. 1F, the spinal spacer 100 includes two openings 116. The opening 116 can be configured to be for placing and maneuvering of the spacer 100 into the vertebrae of the patient. In some embodiments, the opening 116 can be configured to allow placement of the bone graft material. Further, the opening 116 can protrude through the wall of the right side 112 in such a way that it connects the hollow interior 122 with the exterior of the spacer 100. In other embodiments, the opening 116 can be configured to be a groove, which means that the opening 116 does not protrude all the way from the exterior of the spacer 100 to the hollow interior 122. In embodiments having more than one opening 116, the openings can be configured to be symmetrically disposed on the right side 112. As can be understood by one skilled in the art, the openings 116 can be disposed on any side of the spacer 100. Additionally, as illustrated in FIG. 1E, the right side 112 can also include a threaded opening 126 that includes threads 120 configured to accommodate bone screws for further securing of the spacer 100 in the vertebrae of the patient. In FIG. 1E embodiments, the openings 116 are placed symmetrically about the threaded opening 126.

As shown in FIGS. 1D and 1F, the openings 116 can have a length D6 and width D5 and can be disposed a distance D7 away from the center of the threaded opening 126. Opening 126 can have a radius R5. In exemplary embodiments, D5=0.5 mm, D6=0.15 mm, D7=0.09 mm, R5=0.5 mm.

The sides 106 and 108 may have varying degrees convexity and concavity, as illustrated in FIGS. 1B, 1C, 1D, and 1G. Referring to FIG. 1G, the front portion 106 has a curvature radius R8. The back portion 108 has a curvature radius 107. The length of the spacer 100 can be defined as the distance D from the outermost point on the left side 110 to the outermost point on the right side 112. The width of the spacer 100 can be defined as the distance E from the outermost point in the front side 106 to the outermost point on the backside 108. In exemplary embodiments, R7=25 mm, R8=15 mm, D=22 mm, E=10.92 mm. The various curvatures of the spinal spacer 100 can be configured to closely match the shape of the vertebrae discs of the patient. This way, the spinal spacer allows better movement and flexibility of the vertebrae with the spacer installed. As can be understood by one skilled in the art, the sides 108 and 110 may have varying heights. For example, the height of side 108 can be greater than the height of side 110, as illustrated in FIG. 1B. Further, in some embodiments, the height of sides 106, 108, 110, and 112 can vary throughout the device, as desired. For example, the height of at least a portion of the side 106 can be greater than the height of at least a portion of the side 108. The height can also vary within each side 106, 108, 110, and 112. This means that, for example, a portion of the left side 110 can have a lesser height than another portion of the left side 110. Such variation in heights throughout the sides of the spinal spacer 100 can be based on a particular design choice and further configured to accommodate various dimensions of the vertebrae of the patient. Also, the thickness of the walls can vary between the sides 106, 108, 110, and 112. The thickness can also vary within each side 106, 108, 110, and 112. This means that, for example, the thickness of at least a portion of the right side 112 can greater than the thickness of at least another portion of the right side 112.

The openings 116 and 126 can be located anywhere in the sides of the spacer 100. The openings 126 may include threads 120 or any other securing patterns (mechanical locks, hooks, etc.) configured to allow insertion of screws or other devices that secure the spinal spacer 100.

FIGS. 2A-2H illustrate an alternate embodiment of the spinal spacer 200. The spinal spacer 200 is similar to the spinal spacer 100. In the illustrated embodiment, the height of side 210 of the spinal spacer 200 is less than the height of side 110 of spacer 100. In other aspects the two embodiments may be similar.

FIGS. 2A-2H illustrate a spinal spacer 200 that includes a top portion 202, a bottom portion 204, a front side 206, a back side 208, a left side 210, and a right side 212. The front side 206, the back side 208, the left side 210 and the right side 212 may have a varying height, length, thickness, and/or curvature radius, as illustrated in FIGS. 2A-2H.

As shown in FIG. 2A, a combination of the sides 206, 208, 210, and 212 forms a wall that encloses a hollow interior 222. The top portion 202 and the bottom portion 204 include a plurality of teeth 218. Teeth 218 can be configured to be spaced throughout the top portion 202 and the bottom portion 204. As can be understood by one skilled, the teeth 218 can be configured to have variable thickness, height, and width as well as angles of orientation with respect to surfaces of portions 202 and 204. The teeth 218 can be further configured to provide additional support after the spinal spacer 200 is implanted in the vertebrae of the patient. The teeth 218 can reduce movement of the spinal spacer 200 in the vertebrae and create additional friction between the vertebrae and the spacer 200. If more than one spinal spacer 200 is implanted in the vertebrae of the patient, the teeth 218 of one spinal spacer 200 can be configured to interact and/or mate with teeth of another spinal spacer, thereby creating stacked spacers. Such interaction can be useful, when multiple spinal spacers are needed to be implanted into the vertebrae. As shown in FIG. 2H (detail B of the side view of the spinal spacer 200), the teeth 218 can be configured to have a shape of triangular protrusions extending away from the surfaces of the top and bottom portions of the spinal spacer 200. The triangular protrusions can be configured to be right-angled isosceles triangles, as illustrated in detail B of FIG. 2H. As can be understood by one skilled in the art, the triangular protrusions can be any size and shape triangles are not necessarily limited to the right-angled isosceles triangles. Further, the triangular protrusions can be configured to protrude a distance D12 away from the surface (whether top or bottom surfaces) of the spinal spacer 200. The triangular protrusions can also be spaced apart a distance D11, as illustrated in detail B of FIG. 2H. In some embodiments, D12=0.03 millimeters (“mm”) and D11=0.1 mm. As can be understood by one skilled in the art, the teeth 218 can be configured to have any shape, size, or orientation as well as can protrude any distance away from the surfaces of the spinal spacer and can have any distance between them.

Referring back to FIGS. 2A-2C, in some embodiments, the teeth 218 can be configured to be evenly spaced on the top portion 202 and the bottom portion 204. In other embodiments, the teeth 218 can be configured to be spaced in a predetermined order, such as the one shown in FIGS. 2A-C.

FIG. 2B is a bottom view of the spinal spacer 200 shown in FIG. 2A. The teeth 218 are configured to be disposed on the bottom portion 204 in a predetermined order. Specifically, the teeth 218 include a plurality of spacings 235(a, b) and 233(a, b). As illustrated in FIG. 2B, spacings 233a and 235a are configured to be disposed between teeth 218 on the bottom face 204 adjacent the front portion 206 of the spinal spacer 200. The spacings 233b and 235b are configured to be disposed between teeth 218 on the bottom face 204 adjacent the back portion 208 of the spinal spacer 200. Each spacing 233 and 235 is configured to be disposed at predetermined angles with regard to each other. In particular, spacings 233a and 233b are configured to be disposed at angles substantially matching a curvature of the front and back portions 206 and 208 of the spinal spacer 200. Similarly, spacings 235a and 235b are also configured to be disposed at angles substantially matching a curvature of the portions 206, 208. Further, the angular directions of spacings 233(a, b) and spacings 235(a, b) are configured to point away from each other, as illustrated in FIG. 2B. Such disposition allows the spinal spacer to more closely match the shapes and sizes of the vertebrae and accommodate other spinal spacers 200 in the event that spinal spacers are stacked together.

As further illustrated in FIG. 2B, some teeth 218 can have a different length than the other teeth 218. For example, teeth located on the bottom surface 204 adjacent the right side 212 and the left side 210 can be configured to be longer than the teeth located on the bottom surface 204 adjacent the front and back portions 206, 208.

FIG. 2C is a top view of the exemplary spinal spacer 200 shown in FIG. 2A. The top surface 202 also includes a plurality of teeth 218 that can be configured to have a similar structure as shown in FIG. 2H. The teeth 218 can be disposed through the top surface 202 in a similar fashion as their counterparts in the bottom surface 204. The teeth disposition can be substantially symmetrical about a center axis of the spacer 200. As can be understood by one skilled in the art, such symmetrical disposition can be in the top surface 202 as well as in the bottom surface 204 of the spinal spacer 200. Further, as shown in FIGS. 2B and 2C, the wall formed adjacent to the right side 212 of the spinal spacer 200 can be configured to have a greater thickness than the walls formed adjacent to the front portion, back portion, and left side of the spacer 200. In some embodiments, the thicknesses of the front portion, back portion, and left side can be configured to be substantially the same. As illustrated in FIG. 2E, such thicknesses W1 can be on the order of 0.1 mm, whereas thickness W2 of the wall adjacent to the right side 212 can be on order of 0.19 mm. As can be understood by one skilled in the art, these numerical values are provided here for exemplary purposes only and are not intended to limit the present invention in any way. The front portion 206 and the back portion 208 can be further configured to have a convex/concave shape. The convexity/concavity of these portions can be further defined by a radius R1. In exemplary embodiments, R1=25°. The spacings 233 and 235 can be disposed between teeth 218 to substantially match such angular disposition. At least a portion of the teeth 218 can also be disposed along top and bottom surfaces in an angular direction that substantially matches radius R1. As can be understood by one skilled in the art, other radii can be used to define curvatures of the spinal spacer 200.

FIG. 2D is a side view of the exemplary spinal spacer 200 illustrated in FIG. 2A. As illustrated in FIG. 2D, the left side 210 of the spinal spacer 200 can be configured to have a lesser thickness M than the thickness N of the right side 212 of the spinal spacer 200. The right side 212 can be further configured to accommodate an opening 216. In some embodiments, the spacer 200 can be configured to include more than one opening 216. As illustrated in FIG. 2F, the spinal spacer 200 includes two openings 216. The opening 216 can be configured to be for placing and maneuvering of the spacer 200 into the vertebrae of the patient. In some embodiments, the opening 216 can be configured to allow placement of the bone graft material. Further, the opening 216 can protrude through the wall of the right side 212 in such a way that it connects the hollow interior 222 with the exterior of the spacer 200. In other embodiments, the opening 216 can be configured to be a groove, which means that the opening 216 does not protrude all the way from the exterior of the spacer 200 to the hollow interior 222. In embodiments having more than one opening 216, the openings can be configured to be symmetrically disposed on the right side 212. As can be understood by one skilled in the art, the openings 216 can be disposed on any side of the spacer 200. Additionally, as illustrated in FIG. 2E, the right side 212 can also include a threaded opening 226 that includes threads 220 configured to accommodate bone screws for further securing of the spacer 200 in the vertebrae of the patient. In FIG. 2E embodiments, the openings 216 are placed symmetrically about the threaded opening 226.

As shown in FIGS. 2D and 2F, the openings 216 can have a length D6 and width D5 and can be disposed a distance D7 away from the center of the threaded opening 226. Opening 226 can have a radius R5. In exemplary embodiments, D5=0.5 mm, D6=0.15 mm, D7=0.09 mm, R5=0.5 mm.

The sides 206 and 208 may have varying degrees convexity and concavity, as illustrated in FIGS. 2B, 2C, 2D, and 2G. Referring to FIG. 2G, the front portion 206 has a curvature radius R8. The back portion 208 has a curvature radius 207. The length of the spacer 200 can be defined as the distance D from the outermost point on the left side 210 to the outermost point on the right side 212. The width of the spacer 200 can be defined as the distance E from the outermost point in the front side 206 to the outermost point on the backside 208. In exemplary embodiments, R7=25 mm, R8=15 mm, D=22 mm, E=10.92 mm. The various curvatures of the spinal spacer 200 can be configured to closely match the shape of the vertebrae discs of the patient. This way, the spinal spacer allows better movement and flexibility of the vertebrae with the spacer installed. As can be understood by one skilled in the art, the sides 208 and 210 may have varying heights. For example, the height of side 208 can be greater than the height of side 210, as illustrated in FIG. 2B. Further, in some embodiments, the height of sides 206, 208, 210, and 212 can vary throughout the device, as desired. For example, the height of at least a portion of the side 206 can be greater than the height of at least a portion of the side 208. The height can also vary within each side 206, 208, 210, and 212. This means that, for example, a portion of the left side 210 can have a lesser height than another portion of the left side 210. Such variation in heights throughout the sides of the spinal spacer 200 can be based on a particular design choice and further configured to accommodate various dimensions of the vertebrae of the patient. Also, the thickness of the walls can vary between the sides 206, 208, 210, and 212. The thickness can also vary within each side 206, 208, 210, and 212. This means that, for example, the thickness of at least a portion of the right side 212 can greater than the thickness of at least another portion of the right side 212.

The openings 216 and 226 can be located anywhere in the sides of the spacer 200. The openings 226 may include threads 220 or any other securing patterns (mechanical locks, hooks, etc.) configured to allow insertion of screws or other devices that secure the spinal spacer 200.

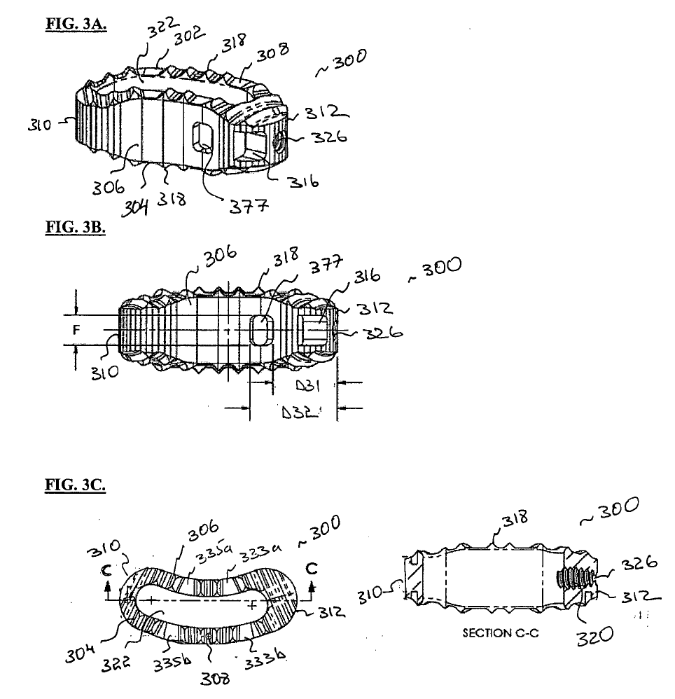

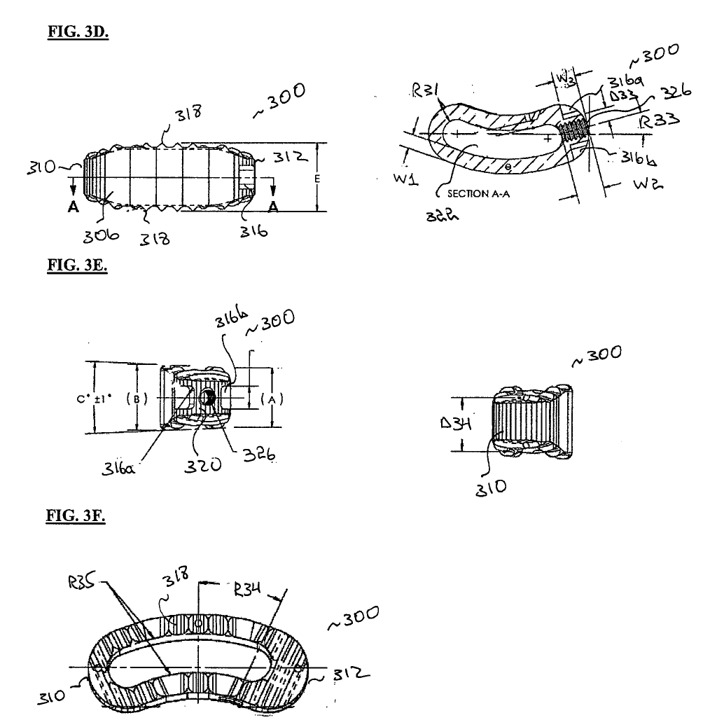

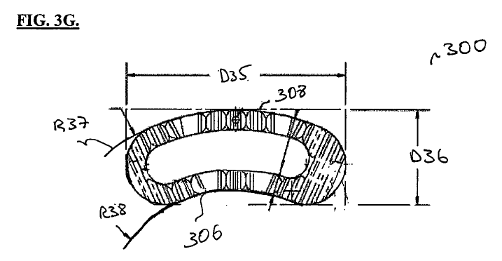

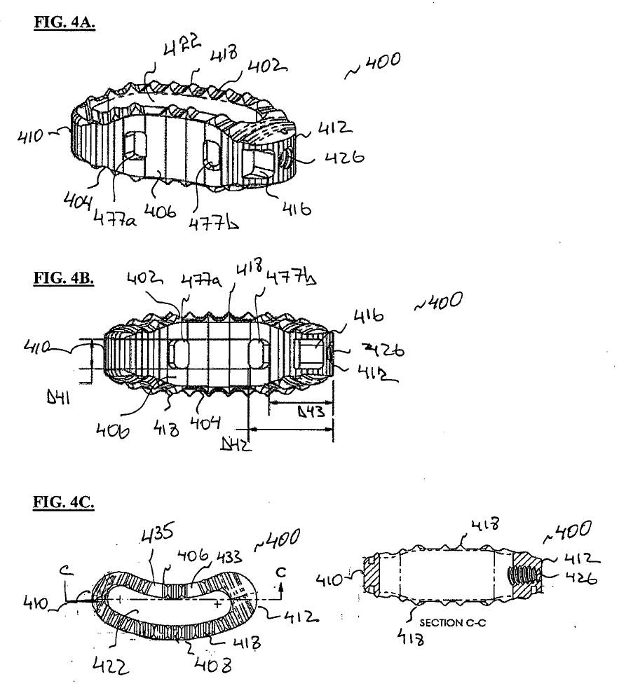

FIGS. 3A-4C illustrate alternate embodiments of the spacers 300 and 400, respectively.

FIGS. 3A-3G illustrate an alternate embodiment of the spinal spacer 300. The spinal spacer 300 is similar to the spinal spacer 100. The spinal spacer 300 includes a top portion 302, a bottom portion 304, a front side 306, a back side 308, a left side 310, and a right side 312. The front side 306, the back side 308, the left side 310 and the right side 312 may have a varying height, length, thickness, and/or curvature radius.

As shown in FIG. 3A, a combination of the sides 306, 308, 310, and 312 forms a wall that encloses a hollow interior 322. The top portion 302 and the bottom portion 304 include a plurality of protrusions or teeth 318 (hereinafter, referred to as “teeth”). Teeth 318 can be configured to be spaced throughout the top portion 302 and the bottom portion 304. As can be understood by one skilled, the teeth 318 can be configured to have variable thickness, height, and width as well as angles of orientation with respect to surfaces of portions 302 and 304. The teeth 318 can be further configured to provide additional support after the spinal spacer 300 is implanted in the vertebrae of the patient. The teeth 318 can reduce movement of the spinal spacer 300 in the vertebrae and create additional friction between the vertebrae and the spacer 300. If more than one spinal spacer 300 is implanted in the vertebrae of the patient, the teeth 318 of one spinal spacer 300 can be configured to interact and/or mate with teeth of another spinal spacer, thereby creating stacked spacers. Such interaction can be useful, when multiple spinal spacers are needed to be implanted into the vertebrae. The teeth 318 can be configured to be similar in structure, shape, size, etc. to the teeth 118 and 218 illustrated with regard to FIGS. 1A-2H above. As can be understood by one skilled in the art, the teeth 318 can be configured to have any shape, size, or orientation as well as can protrude any distance away from the surfaces of the spinal spacer and can have any distance between them.

In some embodiments, the teeth 318 can be configured to be evenly spaced on the top portion 302 and the bottom portion 304, such as shown in FIGS. 4A-4C. In other embodiments, the teeth 318 can be configured to be spaced in a predetermined order, such as the one shown in FIGS. 3A-C.

FIG. 3B is a side view of the spinal spacer 300 and FIG. 3C is a bottom view of the spinal spacer 300 shown in FIG. 3A. The teeth 318 are configured to be disposed on the bottom portion 304 in a predetermined order. Specifically, the teeth 318 include a plurality of spacings 335(a, b) and 333(a, b). The structure, disposition, orientation, and other parameters of the spacings 333 and 335 are similar to the spacings 133 and 135 discussed above.

As further illustrated in FIG. 3C, some teeth 318 can have a different length than the other teeth 318. For example, teeth located on the bottom surface 304 adjacent the right side 312 and the left side 310 can be configured to be longer than the teeth located on the bottom surface 304 adjacent the front and back portions 306, 308.

FIG. 3D is a side view and a cross-sectional view of the exemplary spinal spacer 300 taken at cross-section A-A. As shown in FIGS. 3C and 3D, the wall formed adjacent to the right side 312 of the spinal spacer 300 can be configured to have a greater thickness than the walls formed adjacent to the front portion, back portion, and left side of the spacer 300. In some embodiments, the thicknesses of the front portion, back portion, and left side can be configured to be substantially the same. As illustrated in FIG. 3D, such thicknesses W1 can be on the order of 0.1 mm, whereas thickness W2 of the wall adjacent to the right side 312 can be on order of 0.19 mm. As can be understood by one skilled in the art, these numerical values are provided here for exemplary purposes only and are not intended to limit the present invention in any way. The front portion 306 and the back portion 308 can be further configured to have a convex/concave shape. The convexity/concavity of these portions can be further defined by a radius R31, as illustrated in FIG. 3D. In some embodiments, both sides 312

Referring back to FIG. 3A, the right side 312 can be further configured to accommodate an opening 316. In some embodiments, the spacer 300 can be configured to include more than one opening 316. As illustrated in FIG. 3E, the spinal spacer 300 includes two openings 316. The opening 316 can be configured to be for placing and maneuvering of the spacer 300 into the vertebrae of the patient. In some embodiments, the opening 316 can be configured to allow placement of the bone graft material. Further, the opening 316 can protrude through the wall of the right side 312 in such a way that it connects the hollow interior 322 with the exterior of the spacer 300. In other embodiments, the opening 316 can be configured to be a groove, which means that the opening 316 does not protrude all the way from the exterior of the spacer 300 to the hollow interior 322. In embodiments having more than one opening 316, the openings can be configured to be symmetrically disposed on the right side 312. As can be understood by one skilled in the art, the openings 316 can be disposed on any side of the spacer 300. Additionally, as illustrated in FIGS. 3A and 3E, the right side 312 can also include a threaded opening 326 that includes threads 320 configured to accommodate bone screws for further securing of the spacer 300 in the vertebrae of the patient. In FIG. 3E embodiment, the openings 316 are placed symmetrically about the threaded opening 326. Additionally, the spacer 300 can be configured to include an opening 377 in the front side 306. As illustrated in FIGS. 3A and 3B, the opening 377 is configured to be located a distance D31 from the outer edge of the right side 312. In some embodiments, D31=0.33 inches and the width of the opening 377 as defined by the differences between D32 and D31 is on the order of 0.12 inches. The opening 377 can be configured as a partial protrusion into the front side 306. In some embodiments, the opening 377 can be configured to connect the interior 322 of the spacer 300 to its exterior. The opening 377 can be also configured to accommodate placement and maneuvering of the spacer 300 into the vertebrae of the patient. It can also be configured to allow placement of the bone graft material. As can be understood by one skilled in the art, the spacer 300 can also include more than one opening 377. Such openings 377 can be located anywhere (i.e., front side 406, back side 408, left side 410 and/or right side 412) in the spacer 300. Further, the opening 377 can be located anywhere on any side of the spacer 300. As shown in FIG. 4A, the spacer 400 includes two openings 477 in its front side 406. As can be understood by one skilled in the art, the openings 477 can be of the same size or different sizes.

The sides 306 and 308 may have varying degrees convexity and concavity, as illustrated in FIGS. 3B, 3C, 3D, and 3G. Referring to FIG. 3G, the front portion 306 has a curvature radius R38. The back portion 308 has a curvature radius R37. The length of the spacer 300 can be defined as the distance D35 from the outermost point on the left side 310 to the outermost point on the right side 312. The width of the spacer 300 can be defined as the distance D36 from the outermost point in the front side 306 to the outermost point on the backside 308. In exemplary embodiments, R38=35 mm, R37=15 mm, D35=32 mm, D36=10.92 mm. The various curvatures of the spinal spacer 300 can be configured to closely match the shape of the vertebrae discs of the patient. This way, the spinal spacer allows better movement and flexibility of the vertebrae with the spacer installed. As can be understood by one skilled in the art, the sides 308 and 310 may have varying heights. For example, the height of side 308 can be greater than the height of side 310, as illustrated in FIG. 3B. Further, in some embodiments, the height of sides 306, 308, 310, and 312 can vary throughout the device, as desired. For example, the height of at least a portion of the side 306 can be greater than the height of at least a portion of the side 308. The height can also vary within each side 306, 308, 310, and 312. This means that, for example, a portion of the left side 310 can have a lesser height than another portion of the left side 310. Such variation in heights throughout the sides of the spinal spacer 300 can be based on a particular design choice and further configured to accommodate various dimensions of the vertebrae of the patient. Also, the thickness of the walls can vary between the sides 306, 308, 310, and 312. The thickness can also vary within each side 306, 308, 310, and 312. This means that, for example, the thickness of at least a portion of the right side 312 can greater than the thickness of at least another portion of the right side 312.

As illustrated in FIG. 3G, the front and the back sides of the spacer 300 are concave (this includes interior portions of the front and back sides). In some embodiments, the front side of the spacer can be convex and the back side of the spacer can be concave. In some embodiments, the front side of the spacer can be concave and the back side of the spacer can be convex. In yet other embodiments, both sides can be concave. Further, the interior portions of the front and back sides can be either convex, concave, or any combination of the convex/concave. The convexity/concavity of the interior portions can match the convexity/concavity of the exterior portions of the front and back sides. In some embodiments, the exterior portion of a side can be convex and the interior portion of the side can be concave and vice versa. In yet other embodiments, a side of the spacer can have multiple concave and/or convex regions.

Referring to FIG. 3D, which is a side and cross-sectional view taken at line A-A, the threaded opening 326 is configured to be disposed at an angle R33 inside the spacer 300. Such angle can vary according to a desired configuration of the spacer 300 and in some embodiments can be on the order of 15°. Such angular disposition of the threaded opening 326 can assist a surgeon in placement and maneuvering of the spacer 300 during installation into the vertebrae of the patient. FIG. 3C is a side view and a cross-sectional view taken at line C-C that further illustrates threaded opening 326 having threads 320. Referring back to FIG. 3D, the center of threaded opening 326 can be located distance D33 away from the edge of the opening 316. The depth of the opening 316 can be configured to be a distance W3 from the edge of the right side 312. In some embodiments, D33=0.09 inches, W3=0.15 inches. As can be understood by one skilled in the art, the present invention is not limited to these dimensions. The openings 316 and 326 can be located anywhere in the sides of the spacer 300. The openings 326 may include threads 320 or any other securing patterns (mechanical locks, hooks, etc.) configured to allow insertion of screws or other devices that secure the spinal spacer 300.

FIGS. 4A-4C illustrate spacer 400 that is similar to the spacer 300 illustrated in FIGS. 3A-3G. One of the differences between spacers 300 and 400 is that spacer 400 includes two openings 477 as opposed to one.

The spinal spacer can be manufactured from a biologically accepted inert material, such as PEEK (Polyetheretherketone). The spacer can be configured to be implanted between the vertebrae for treating degenerative or ruptured discs and/or for replacing damaged vertebral bodies. As stated above, the spacer can be configured to be used singularly or in a stacked combination to fill differently sized evacuated spaces. Each spacer can be particularly shaped and sized for its particular application.

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

What is claimed:

1. A spinal spacer, comprising:
a wall configured to enclose a hollow interior, wherein said wall is further configured to have a top portion and a bottom portion;
said top and bottom portions are configured to include a plurality of projections configured to protrude away from said top and bottom portions;
wherein said wall is further configured to
have at least one curved portion; and
include a plurality of grooves and an opening.

2. The spinal spacer according to claim 1, wherein said wall is further configured to have at least one concave portion and at least one convex portion.

3. The spinal spacer according to claim 1, wherein said wall includes a plurality of concave portions.

4. The spinal spacer according to claim 1, wherein said wall includes a plurality of convex portions.

5. The spinal spacer according to claim 1, wherein said wall is configured to have at least one portion having a greater height than at least another portion.

6. The spinal spacer according to claim 1, wherein said opening is configured to protrude from an exterior of the spinal spacer to said hollow interior and is further configured to include threads.

7. The spinal spacer according to claim 1, wherein said protrusions are configured to evenly space out throughout said top and bottom portions.

8. The spinal spacer according to claim 1, wherein said protrusions are configured to randomly space out throughout said top and bottom portions.

9. The spinal spacer according to claim 1, wherein said grooves are configured to allow placement of tools and maneuvering of the spinal spacer.

10. The spinal spacer according to claim 1, wherein said protrusions are configured to have a shape of a right isosceles triangle.

11. The spinal spacer according to claim 1, wherein said top and bottom portions include a plurality of spacings between said protrusions;
said spacings are configured to be aligned with one another along said curved portion of said wall.

12. The spinal spacer according to claim 1, wherein said wall is configured to have a variable thickness.

13. The spinal spacer according to claim 12, wherein said wall is configured to have at least one portion having a greater thickness than at least another portion.

14. The spinal spacer according to claim 1, wherein the spacer is manufactured from a biologically inert material.

15. A spinal spacer assembly, comprising:
a spinal spacer having:
a wall having two sides, a front portion, and a back portion, wherein said front and back portions are configured to be disposed between said two sides;
said front and back portions are configured to include have at least one curved portion;
at least one side of said two sides includes at least one groove and at least one opening;
said two sides and said front and back portions are configured to enclose a hollow interior;
said wall is further configured to have a top surface and a bottom surface; and
said top and bottom surfaces are configured to include a plurality of protrusions configured to protrude away from said top and bottom portions.

16. The spinal spacer assembly according to claim 15, wherein at least one of said front and back portions is concave and at least another portion is convex.

17. The spinal spacer assembly according to claim 15, wherein said wall includes a plurality of concave portions.

18. The spinal spacer assembly according to claim 15, wherein said wall includes a plurality of convex portions.

19. The spinal spacer assembly according to claim 15, wherein at least a portion of one of said two sides, said front portion, and said back portion is configured to have at least one portion having a greater height than at least another portion of said two sides, said front portion, and said back portion.

20. The spinal spacer assembly according to claim 15, wherein said opening is configured to protrude from an exterior of the spinal spacer to said hollow interior and is further configured to include threads.

21. The spinal spacer assembly according to claim 15, wherein said protrusions are configured to have a shape of a right isosceles triangle.

22. The spinal spacer assembly according to claim 15, wherein said wall is configured to have variable thickness.

23. The spinal spacer assembly according to claim 22, wherein at least a portion of one of said two sides, said front portion, and said back portion is configured to have at least one portion having a greater thickness than at least another portion of said two sides, said front portion, and said back portion.

24. The spinal spacer assembly according to claim 15, wherein the spacer is manufactured from a biologically inert material.

25. The spinal spacer assembly according to claim 15, further comprising a plurality of said spinal spacers.
